(19) United States
(12) Patent Application Publication
(10) Pub. No.: US 2016/0000732 A1
(43) Pub. Date: Jan. 7, 2016

(54) ORAL PHARMACEUTICAL COMPOSITIONS OF OSPEMIFENE

(71) Applicant: CADILA HEALTHCARE LIMITED, Ahmedabad (IN)

(72) Inventors: Sushrut Krishnaji Kulkarni, Ahmedabad (IN); Pavak Rajnikant Mehta, Ahmedabad (IN); Ritesh Kapoor, Ahmedabad (IN)

(73) Assignee: CADILA HEALTHCARE LIMITED, Ahmedabad (IN)

(21) Appl. No.: 14/755,166

(22) Filed: Jun. 30, 2015

(30) Foreign Application Priority Data
Jul. 2, 2014 (IN) 2145/MUM/2014

(51) Int. Cl. A61K 31/085; A61J 3/00; A61K 9/28; A61K 9/20; A61K 9/00; A61K 9/16

(52) U.S. Cl. CPC A61K 31/085; A61K 9/0053; A61K 9/1641; A61K 9/1617; A61K 9/284; A61K 9/2095; A61K 9/1682; A61J 3/005

(57) ABSTRACT

The present invention relates to pharmaceutical compositions comprising ospemifene or a pharmaceutically acceptable salt thereof having an average particle size of more than 20 microns and one or more pharmaceutically acceptable excipients comprising at least one solubility enhancing agent. The compositions of the invention may be advantageously used for the treatment or prevention of atrophy-related diseases or disorders in women, especially in women during or after the menopause.

### FIELD OF THE INVENTION

The present invention relates to pharmaceutical compositions comprising ospemifene or a pharmaceutically acceptable salt thereof having an average particle size of more than 20 microns and one or more pharmaceutically acceptable excipients comprising at least one solubility enhancing agent. The compositions of the invention may be advantageously used for the treatment or prevention of atrophy-related diseases or disorders in women, especially in women during or after the menopause.

### BACKGROUND OF THE INVENTION

Ospemifene is known as 2-[4-(4-chloro-1,2-diphenylbut-1-enyl)phenoxy]ethanol and it is the Z-isomer of the compound of following structural formula:

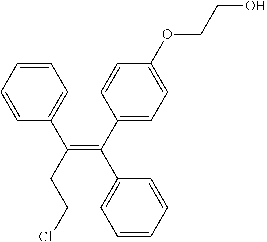 [SMILES: OCCOc1ccc(/C(=C(/CCCl)c2ccccc2)c2ccccc2)cc1]

It is one of the main metabolites of toremifene, and is known to be an estrogen agonist and antagonist as described in Kangas, 1990; PCT Publication Nos. WO 1996/07402 and WO 1997/32574. The compound is also called (deaminohydroxy)toremifene and it is also known under the code FC-1271a. Ospemifene has relatively weak estrogenic and antiestrogenic effects in the classical hormonal tests as described in Kangas, 1990.

According to PCT Publication Nos. WO 1996/07402 and WO 1997/32574, ospemifene has anti-osteoporosis actions and it decreases total and LDL cholesterol levels in both experimental models and in human volunteers.

The use of ospemifene, a selective estrogen receptor modulator (SERM) for the treatment of certain climacteric disorders in postmenopausal women, namely vaginal dryness and sexual dysfunction, is disclosed in PCT Publication No. WO 2002/07718.

Ospemifene belongs to class II category of the biopharmaceutical classification system. The compound is highly lipophilic and its relatively low aqueous solubility makes it difficult to provide a dosage form. Therefore, new highly soluble and bioavailable compositions of ospemifene are needed.

PCT Publication No. WO 2005/079777 relates to a solid drug composition comprising granulates containing a therapeutically active compound, which is ospemifene. The application discloses that particle size of ospemifene in the granulates is important in order to get a good dissolution wherein preferably 90% of the drug substance shall have a particle size less than 50 micrometer, and 50% of the drug substance shall have a particle size less than 15 micrometer.

The current invention provides a novel immediate release composition of ospemifene for oral administration wherein ospemifene or a pharmaceutically acceptable salt thereof has an average particle size of more than 20 microns, preferably more than 25 microns, thus overcoming the particle size restriction of the prior art composition.

### SUMMARY OF THE INVENTION

In one general aspect, there is provided an oral pharmaceutical composition comprising:

a) ospemifene or a pharmaceutically acceptable salt thereof having an average particle size of more than 20 microns; and

b) one or more pharmaceutically acceptable excipients comprising at least one solubility enhancing agent,

wherein the composition is prepared using a granulation technique.

In another general aspect, there is provided a pharmaceutical composition, wherein the composition comprises about 20 mg to 100 mg of ospemifene, more preferably about 30 mg to 90 mg of ospemifene.

In another general aspect, there is provided a pharmaceutical composition of ospemifene, wherein the at least one solubility enhancing agent is present intragranularly.

In another general aspect, there is provided a pharmaceutical composition of ospemifene, wherein the ospemifene has an average particle size more than 25 microns.

In another general aspect, there is provided a pharmaceutical composition of ospemifene, wherein the solubility enhancing agent comprises about 0.1 to about 6% by weight of the intra-granular composition.

In another general aspect, there is provided a pharmaceutical composition of ospemifene, wherein the composition is in the form of a tablet, a capsule, pellets, granules, a sachet or sprinkles.

Embodiments of the pharmaceutical composition may include one or more of the following features. For example, the pharmaceutical composition may further comprise one or more pharmaceutically acceptable excipients. The pharmaceutically acceptable excipients may comprise one or more of binders, fillers, complexing agents, enhancing agents, disintegrants, lubricants, glidants, sweetening agents, anti-tacking agents and the like.

In another general aspect, there is provided a pharmaceutical composition of ospemifene, wherein the solubility enhancing agent comprises surface active agent comprising sodium lauryl sulfate, polysorbate 80, benzyl alcohol, sorbitan monolaurate, poloxamer 407 or combinations thereof.

In another general aspect, there is provided a process for preparing a pharmaceutical composition, wherein the process comprises the steps of:

a) preparing a solution or dispersion of ospemifene with at least one solubility enhancing agent;

b) drying the resultant solution/dispersion;

c) adding one or more pharmaceutically acceptable excipients to obtain a blend; and

d) compressing the blend into tablet.

In another general aspect, there is provided a process for preparing a pharmaceutical composition, wherein the process comprises the steps of:

a) preparing a solution or dispersion of ospemifene with at least one solubility enhancing agent and one or more pharmaceutically acceptable excipients;

b) spraying the ospemifene solution or dispersion over a carrier bed to obtain granules;

c) drying the granules;

d) blending the dried granules with one or more pharmaceutically acceptable excipients;

e) lubricating the blend; and

f) compressing the blend into tablet.

In another general aspect, there is provided a process for preparing a pharmaceutical composition, wherein the process comprises the steps of:

a) mixing ospemifene with one or more pharmaceutically acceptable excipients;

b) compacting the mixture to form slugs;

c) milling the slugs to form mixture of granules and fines;

d) granulating the milled mixture with one or more pharmaceutically acceptable excipients using a binder solution comprising at least one solubility enhancing agent;

e) drying the resulting granules;

f) sizing and milling the dried granules;

g) blending the granules with one or more pharmaceutically acceptable excipients;

h) compressing the blend into tablets; and

i) optionally coating the tablets.

Embodiments of the pharmaceutical composition may include one or more of the following features. For example, the pharmaceutical composition may further comprise one or more pharmaceutically acceptable excipients. The pharmaceutically acceptable excipients may comprise one or more of binders, fillers, complexing agents, enhancing agents, disintegrants, lubricants, glidants, sweetening agents, anti-tacking agents and the like.

In another general aspect, there is provided a method of treatment or prevention of atrophy-related diseases or disorders in women, especially in women during or after the menopause, comprising orally administering to the subject the pharmaceutical composition of ospemifene as per the invention.

The details of one or more embodiments of the invention are set forth in the description below. Other features of the invention will be apparent from the description.

### DETAILED DESCRIPTION OF THE INVENTION

The present invention provides an immediate release composition of ospemifene for oral administration, wherein ospemifene or a pharmaceutically acceptable salt thereof has an average particle size of more than 20 microns.

It has now surprisingly been found that ospemifene compositions can be prepared, wherein ospemifene or a pharmaceutically acceptable salt thereof has an average particle size of more than 20 microns and wherein at least about 80% of the composition is dissolved within 30 minutes after subjecting said composition to dissolution testing.

The term “ospemifene” includes a geometric isomer, a polymorph, a stereoisomer, a pharmaceutically acceptable salt, an ester thereof or a metabolite thereof.

The term “particle size” refers to the particle diameter, or in case the particles are not spherical, to the largest extension in one direction of the particle.

The term “immediate release composition” refers to a composition that releases greater than or equal to about 80% of ospemifene in less than or equal to about 1 hour.

Excipients in the granules are also called intra-granular excipients. When the granules are formulated to the final compositions, further excipients are added. Excipients outside the granules are called extra-granular excipients.

Carriers useful in the present invention comprise one or more pharmaceutically acceptable excipients. It may also comprise cellulose spheres, silicon dioxide, starch and sugar spheres. The inert carrier is present in an amount of from about 5% to about 90% by weight.

For the purposes of this application, an “enhancing agent” (an enhancer), is defined as any non-pharmaceutically active ingredient that improves the efficacy and therapeutic potential of a composition.

The composition according to the present invention comprises ospemifene or a pharmaceutically acceptable salt thereof, wherein at least 10% of ospemifene has a particle size (d₁₀) of more than 10 microns, preferably more than 20 microns and 50% of ospemifene has a particle size (d₅₀) of more than 20 microns, more preferably more than 25 microns, preferably from 25 to about 75 microns, more preferably from about 30 to about 70 microns. Further, 90% of ospemifene may have a particle size (d₉₀) more than 50 microns, preferably more than 100 microns, more preferably more than 150 microns.

The pharmaceutically acceptable excipients comprise one or more of binders, fillers, complexing agents, enhancing agents, disintegrants, lubricants, glidants, anti-tacking agents, plasticizers and the like.

Suitable binders may include one or more of carbomers, dextrin, ethyl cellulose, shellac, zein, gelatin, polymethacrylates (eg. eudragit), pregelatinized starch, sodium alginate, gums, synthetic resins, silicic acid, hydrophilic polymers and the like. The binders may be used in dry form or as a binder solution in aqueous or non-aqueous solvents.

The term “hydrophilic polymer” may include polymers with polar groups. Examples of polar groups are hydroxy, amino, carboxy, carbonyl, ethers, esters, and sulfonates. Examples of suitable hydrophilic polymers are cellulose derivatives, in particular hydrophilic derivatives of the cellulose (e.g. hydroxypropyl methylcellulose (HPMC), hydroxypropyl cellulose (HPC), carboxymethylcellulose or their sodium or calcium salt, hydroxyethylcellulose), polyvinylpyrrolidone, preferably with a molecular weight of from 10,000 to 60,000 g/mol, copolymers of PVP, preferably co-polymers comprising vinylpyrrolidone and vinylacetate units (e.g. povidone, VA64, BASF), preferably with a molecular weight between 40,000 and 70,000 g/mol, polyoxyethylene) alkyl ether, polyethylene glycol, co-block polymers of ethylene oxide, and propylene oxide (poloxamer, pluronic), derivatives of polymethacrylates, polyvinyl alcohol, polyvinyl alcohol derivatives, polyethylene glycol, and polyethylene glycol derivatives.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like. The filler may be present in an amount of 5 to 80% by weight of the composition.

Suitable complexing agents may include one or more of benzoates, hydroxybenzoates, amines, amides or polyamines, such as polyvinylpyrrolidones, polyamines e.g. polyvinyl amines and polyallylamines, polyethylene imines, polyvinyl pyridine, and polylysine, oligo- and polysaccharides such as cyclodextrins and their derivatives e.g. hydroxypropyl-beta-cyclodextrin, beta-cyclodextrin, gamma-cyclodextrin, and alpha-cyclodextrin, aminopolysaccharides such as chitosan, polyoxoalkylenes, or polysiloxanes.

Suitable enhancing agents may include one or more of solubility enhancing agents, dissolution enhancing agents, absorption enhancing agents, penetration enhancing agents, surface active agents and stabilizing agents. The representative, but non-limiting examples of these compounds are Vitamin E TPGS, amino acids such as glutamic acid and glycine, sorbitol, mannose, amylose, maltose, mannitol, lactose, sucrose, glucose, xylitose, dextrins such as maltodextrin, Cremophor RH40 (glycerol-polyethylene glycol oxystearate), Gelucire 50/13 (PEG-32 glyceryl palmitostearate), sodium lauryl sulfate, Tween 80 (polyoxyethylene sorbitan monooleate or polysorbate 80), benzyl alcohol, Span 20 (sorbitan monolaurate), Poloxamer 407, polyethylene glycols, such as PEG3350; polyvinylpyrrolidones such as PVP K25, polyvinylalcohols, polyalcohols, oleic acid, Capmul GMO (glyceryl monooleate), sodium benzoate, cetyl alcohol, sucrose stearate, crospovidone, sodium starch glycolate, crosscarmellose sodium, carboxymethylcellulose, starch, pregelatinized starch, HPMC, substituted hydroxypropylcellulose, microcrystalline cellulose, sodium bicarbonate, calcium citrate, sodium docusate, and menthol, among others. Enhancers may be combined to achieve multiple enhancement effects, for example, solubility enhancement combined with permeability enhancement, or to provide a synergistic effect to achieve greater and more efficient enhancement. The enhancing agents may also serve more than one function. For example the surface active agents may also function as solubility enhancing agents. The enhancing agents may comprise from about 0.1 to about 50% by weight of the intra-granular composition. For example surface active agents like sodium lauryl sulfate, Tween 80 (polyoxyethylene sorbitan monooleate or polysorbate 80), benzyl alcohol, Span 20 (sorbitan monolaurate) or Poloxamer 407 may comprise from about 0.1 to about 10% by weight of the composition, more preferably from about 0.1 to about 5% by weight of the composition, about 0.1 to about 6% by weight of the intra-granular composition, more preferably from about 0.1 to about 2.5% by weight of the intra-granular composition.

Suitable disintegrants may include one or more of crosscarmellose sodium, sodium starch glycolate, low substituted hydroxylpropyl cellulose(L hydroxylpropyl cellulose), pregelatinized starch, sodium carboxymethyl cellulose, cross-linked polyvinylpyrrolidone and the like.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like. It would be appreciated that a person skilled in the art is cognizant of the fact that lubricant, glidant or anti-tacking agent may be used interchangeably. The lubricant, glidant or anti-tacking agent may be present in an amount ranging from 0.1% to 15% w/w of the composition, preferably 0.1% to 10%, even more preferably 0.1% to 5% by weight of the composition.

Suitable anti-tacking agents may include one or more of talc, magnesium stearate and the like.

Suitable plasticizers may include one or more of triacetin, diethyl phthalate, tributyl sebacate, polyethylene glycol and the like.

In one embodiment, the pharmaceutical compositions of the invention may comprise about 30 mg to about 90 mg of ospemifene, a solubility enhancing agent and one or more other pharmaceutically acceptable excipients, wherein ospemifene has an average particle size of more than 20 microns, preferably more than 25 microns.

The pharmaceutical compositions as described herein may be prepared by processes known to the person having ordinary skill in the art of pharmaceutical technology such as direct compression, wet granulation, dry granulation, melt granulation, extrusion, co-milling, homogenization, spray granulation, spray coating, freeze drying, spray drying and solvent evaporation and the like. The composition may also be prepared by dry granulation of the active with one or more pharmaceutically acceptable excipients followed by wet granulation with one or more pharmaceutically acceptable excipients.

The co-milling process comprises the use of several miffing techniques known in the art. Suitable dispersion mills include a ball mill, an attrition mill, a vibratory mill, and media mills such as a sand mill and a bead mill. Media milling is a high energy milling process. Ospemifene with other pharmaceutically acceptable excipients is placed in a reservoir and recirculated in a chamber containing media and a rotating shaft/impeller.

Exemplary homogenization method comprises dispersing particles of ospemifene with one or more pharmaceutically acceptable excipients, in a liquid dispersion medium, followed by subjecting the dispersion to homogenization to reduce the particle size of Ospemifene to the desired effective particle size.

In another embodiment, the pharmaceutical compositions of the invention may comprise ospemifene having an average particle size of more than 20 microns, preferably more than 25 microns, wherein ospemifene may be milled with one or more pharmaceutically acceptable excipients, granulated using a binder solution. The granules may be dried and finally compressed using suitable tooling.

In another embodiment, the pharmaceutical compositions of the invention may comprise ospemifene having d₉₀ of more than 50 microns, preferably more than 150 microns, wherein ospemifene may be mixed with a solubility enhancing agent and may be further processed using suitable tooling.

In another embodiment, the pharmaceutical compositions of the invention may comprise ospemifene having an average particle size of more than 20 microns, preferably more than 25 microns, wherein ospemifene may be dissolved in a solution comprising one or more pharmaceutically acceptable excipients, adsorbed on a carrier bed, dried, blended with one or more one or more pharmaceutically acceptable excipients and may be compressed using suitable tooling.

In one embodiment, there is provided a pharmaceutical composition prepared by the process comprising:

a) preparing a solution or dispersion of ospemifene with at least one solubility enhancing agent, and a suitable solvent;

b) drying the resultant solution/dispersion;

c) adding one or more pharmaceutically acceptable excipients;

d) granulating the material of step—c);

e) lubricating the granules obtained;

f) compressing the granules into tablets; and

g) optionally coating the tablets obtained.

In another embodiment, there is provided a pharmaceutical composition prepared by the process comprising:

a) mixing ospemifene with at least one solubility enhancing agent and a solvent;

b) mixing with a binder solution prepared separately to provide a dispersion;

c) spraying the dispersion onto a carrier bed to obtain coated carrier particles,

d) drying the particles;

e) optionally coating the particles; and

f) compressing the particles to obtain a tablet or filling the particles into a capsule.

In another embodiment, there is provided a pharmaceutical composition prepared by the process comprising:

a) preparing a solution or dispersion of ospemifene with at least one solubility enhancing agent and one or more pharmaceutically acceptable excipients;

b) spraying the ospemifene solution or dispersion over a carrier bed comprising one or more pharmaceutically acceptable excipients to obtain granules;

c) drying the granules;

d) milling the dried granules in a co-mill;

e) blending the milled granules with one or more pharmaceutically acceptable excipients;

f) lubricating the blend and compressing into tablets; and

g) optionally coating the tablets.

In another embodiment, there is provided a pharmaceutical composition prepared by the process comprising:

a) preparing a solution or dispersion of ospemifene with at least one solubility enhancing agent and one or more pharmaceutically acceptable excipients;

b) spray drying the ospemifene solution or dispersion to obtain granules;

d) milling the dried granules in a co-mill;

e) blending the milled granules with one or more pharmaceutically acceptable excipients;

f) lubricating the blend and compressing into tablets; and

g) optionally coating the tablets.

In yet another embodiment, there is provided a pharmaceutical composition prepared by the process comprising:

a) mixing ospemifene with one or more pharmaceutically acceptable excipients comprising at least one solubility enhancing agent;

b) granulating the mixture with one or more pharmaceutically acceptable excipients using a binder solution;

c) drying the resulting granules;

d) milling and sizing the dried granules;

e) blending the milled granules with other pharmaceutically acceptable excipients;

f) lubricating the blend and compressing into tablets; and

g) optionally coating the tablets.

In yet another embodiment, there is provided a pharmaceutical composition prepared by the process comprising:

a) mixing ospemifene with one or more pharmaceutically acceptable excipients comprising at least solubility enhancing agent;

b) dry blending the mixture;

c) dry granulating the mixture using roller compaction;

d) milling the roller compacted mixture using a co-mill;

e) granulating the milled mixture with one or more pharmaceutically acceptable excipients using a binder solution;

f) drying the resulting granules;

g) sizing and milling the dried granules;

h) blending the granules with one or more pharmaceutically acceptable excipients;

i)) lubricating the blend and compressing into tablets; and

j) optionally coating the tablets.

In yet another embodiment, there is provided a pharmaceutical composition prepared by the process comprising:

a) mixing ospemifene with one or more pharmaceutically acceptable excipients comprising at least one solubility enhancing agent;

b) dry blending the mixture;

c) dry granulating the mixture using roller compaction;

d) milling the roller compacted mixture using a co-mill;

e) blending the granules with one or more pharmaceutically acceptable excipients;

f) lubricating the blend and compressing into tablets; and

g) optionally coating the tablets.

In yet another embodiment, there is provided a pharmaceutical composition prepared by the process comprising:

a) mixing ospemifene with one or more pharmaceutically acceptable excipients;

b) dry granulating the mixture;

c) milling the granulated mixture;

d) granulating the milled mixture with one or more pharmaceutically acceptable excipients using a binder solution comprising at least one solubility enhancing agent;

e) drying the resulting granules;

f) sizing and milling the dried granules;

g) blending the granules with one or more pharmaceutically acceptable excipients;

h) compressing the blend into tablets; and

i) optionally coating the tablets.

In another embodiment, there is provided a pharmaceutical composition prepared by the process comprising:

a) milling ospemifene with at least one solubility enhancing agent and one or more pharmaceutically acceptable excipients using a high energy mill to obtain a nanosuspension;

b) granulating the milled particles; and

c) optionally processing the granules using suitable tooling to obtain the composition.

The composition of the present invention can be additionally coated with an over-coat. The over-coat can be a moisture barrier coat, a protection coat, a seal coat, a taste-masking coat, a flavor coat, a polish coat, a color coat, or any other cosmetic coat that does not interfere with the release of the active compound or the enhancing agent. Suitable coating materials for such an over-coat are known in the art, and include, but are not limited to, cellulosic polymers such as hydroxypropyl methylcellulose, hydroxypropylcellulose and microcrystalline cellulose, or combinations thereof (for example various Opadry® coating materials).

The compositions according to the present invention is useful in any application of ospemifene, especially when the compound is used for treatment or prevention of osteoporosis or for treatment or prevention of symptoms related to skin atrophy, or to epithelial or mucosal atrophy.

A particular form of atrophy which can be inhibited by administering of ospemifene is urogenital atrophy. Symptoms related to urogenital atrophy can be divided in two subgroups: urinary symptoms and vaginal symptoms. As examples of urinary symptoms can be mentioned micturation disorders, dysuria, hematuria, urinary frequency, sensation of urgency, urinary tract infections, urinary tract inflammation, nocturia, urinary incontinence, urge incontinence and involuntary urinary leakage.

As examples of vaginal symptoms can be mentioned irritation, itching, burning, maladorous discharge, infection, leukorrhea, vulvar pruritus, feeling of pressure and postcoital bleeding.

In another embodiment, the pharmaceutical compositions of ospemifene provide for a relative Cₘₐₓ in the range of 80% to 125%, as compared to the same amount of ospemifene administered as the currently marketed immediate release composition (OSPHENA®).

In yet another general embodiment, a pharmaceutical composition comprising ospemifene, wherein the composition retains at least 95% of the potency of ospemifene in the pharmaceutical composition after storage at 40° C. and 75% relative humidity for at least three months.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

TABLE 1

| | | |
|---|---|---|
| S.N. | Ingredient | % w/w |

Part A (dry granulation and milling)

| | | |
|---|---|---|
| 1 | Ospemifene | 24.49 |
| 2 | Anhydrous lactose | 32.65 |
| 3 | Lactose monohydrate | 7.35 |
| 4 | Cellulose, Microcrystalline | 6.53 |
| 5 | Crospovidone | 3.27 |
| 6 | Silica Colloidal anhydrous | 0.82 |
| 7 | Sodium stearyl fumarate | 0.82 |

Part B (Top Spray Granulation)

| | | |
|---|---|---|
| 8 | Povidone K-30 | 4.90 |
| 9 | Polysorbate 80 | 1.22 |
| 10 | Isopropyl alcohol | q.s |
| 11 | Purified water | q.s |

Part C (Extra Granular ingredients)

| | | |
|---|---|---|
| 12 | Anhydrous lactose | 11.84 |
| 13 | Crospovidone | 2.45 |
| 14 | Silica Colloidal anhydrous | 0.41 |
| 15 | Sodium Stearyl Fumarate | 1.22 |

Part 4 (Coating composition)

| | | |
|---|---|---|
| 16 | Opadry White | 2.04 |
| 17 | Purified Water | q.s |

Process:

Ospemifene (average particle size more than 20 microns), anhydrous lactose, lactose monohydrate, cellulose microcrystalline, crospovidone and silica colloidal anhydrous were co-sifted and blended in a conta-blender and lubricated with sodium stearyl fumarate. The lubricated blend was roller compacted and subsequently milled using appropriate sieve. Granulate the milled material using top spray technique using a binder solution of Povidone and Polysorbate 80 in isopropyl alcohol and water. The obtained granules were dried at an appropriate inlet temperature. The dried granules were sized by sifting and milling. The sized granules were blended with anhydrous lactose, crospovidone and silica colloidal anhydrous and lubricated using sodium stearyl fumarate. The lubricated blend was then compressed into tablets. The tablets were then coated with a dispersion of opadry® in water.

Dissolution Data for Composition of Example 1:

TABLE 1a

| Dissolution | | |
|---|---|---|
| conditions | Time (min) | % drug release |
| Purified water + | 5 | 28.2 |
| 2.0% SDS (Sodium | 10 | 62.3 |
| dodecyl sulphate), | 15 | 77.8 |
| 900 ml, | 20 | 86.8 |
| paddle, | 30 | 93.4 |
| 50 rpm | 45 | 96.6 |

### EXAMPLE 2

TABLE 2

| | | |
|---|---|---|
| S.N | Ingredient | % w/w |

Part A

| | | |
|---|---|---|
| 1. | Ospemifene | 20-30 |
| 2. | Lactose anhydrous | 40-70 |
| 3. | Crospoyidone | 0-8 |
| 4. | Sodium lauryl sulphate | 2-5 |

Part B

| | | |
|---|---|---|
| 5. | Crospoyidone | 3-15 |

Part C

| | | |
|---|---|---|
| 6. | Hydroxypropyl methylcellulose | 3-10 |
| 7. | Water | q.s |

Part D

| | | |
|---|---|---|
| 8. | Lactose | 0-20 |
| 9. | Crospovidone | 0-8 |
| 10. | Magnesium stearate | 0.5-3 |
| 11. | Talc | 0.5-3 |

Part E

| | | |
|---|---|---|
| 12. | Opadry white | 1-3 |
| 13. | Water | q.s |

Process:

Ospemifene (average particle size more than 25 microns), lactose anhydrous, crospovidone and sodium lauryl sulphate were co-milled and then blended with crospovidone. The blend was granulated using a binder solution prepared by dissolving hydroxypropyl methylcellulose in water in a rotary mixer granulator. The granules were dried, screened and blended with lactose, crospovidone, magnesium stearate and talc. The blend was compressed using suitable tooling to obtain tablet. The tablet was then coated with a dispersion of opadry® in water.

### EXAMPLE 3

TABLE 3

| | | |
|---|---|---|
| S.N | Ingredient | % w/w |

Part A

| | | |
|---|---|---|
| 1. | Ospemifene | 20-30 |
| 2. | Lactose anhydrous | 40-70 |
| 3. | Sodium lauryl sulphate | 2-5 |
| 4. | Water | q.s |

Part B

| | | |
|---|---|---|
| 5. | Mannitol | 10-20 |
| 6. | Crospovidone | 3-15 |

Part C

| | | |
|---|---|---|
| 7. | Hydroxypropyl methylcellulose | 3-10 |
| 8. | Water | q.s |

Part D

| | | |
|---|---|---|
| 9. | Crospovidone | 3-8 |
| 10. | Magnesium stearate | 0.5-3 |
| 11. | Talc | 0.5-3 |

Part E

| | | |
|---|---|---|
| 12. | Opadry white | 1-3 |
| 13. | Water | q.s |

Process:

Ospemifene, lactose anhydrous and sodium lauryl sulphate were dispersed in water and homogenized. Hydroxypropyl methylcellulose was dissolved in water to form a solution which was then mixed with the previously formed ospemifene dispersion. The dispersion was then added to a blend of mannitol and crospovidone in a fluid bed processor to obtain granules. The granules were dried and blended with crospovidone, magnesium stearate and talc. The blend was compressed using suitable tooling to obtain tablet. The tablet was then coated with a dispersion of opadry® in water.

### EXAMPLE 4

TABLE 4

| | | |
|---|---|---|
| S.N | Ingredient | % w/w |

Part A

| | | |
|---|---|---|
| 1. | Ospemifene | 20-30 |
| 2. | Hydroxypropyl cellulose | 3-10 |
| 3. | Poloxamer | 2-5 |
| 4. | Acetone | q.s |

Part B

| | | |
|---|---|---|
| 5. | Lactose | 40-70 |
| 6. | Sodium starch glycolate | 3-8 |

Part C

| | | |
|---|---|---|
| 7. | Microcrystalline cellulose | 0-20 |
| 8. | Crospovidone | 0-8 |
| 9. | Magnesium stearate | 0.5-3 |
| 10. | Colloidal silicon dioxide | 0.5-3 |

Part D

| | | |
|---|---|---|
| 11. | Opadry ® | 1-3 |
| 12. | Water | q.s |

Process:

Ospemifene (average particle size more than 30 microns), hydroxypropyl cellulose and poloxamer were dissolved in acetone. The solution was then adsorbed on a blend of lactose and sodium starch glycolate using top spray granulation to obtain granules. The granules were dried and blended with microcrystalline cellulose, crospovidone, magnesium stearate and colloidal silicon dioxide. The blend was compressed using suitable tooling to obtain tablet. The tablet was then coated with a dispersion of opadry® in water.

1. An oral pharmaceutical composition comprising:
a) ospemifene or a pharmaceutically acceptable salt thereof having an average particle size of more than 20 microns; and
b) one or more pharmaceutically acceptable excipients comprising at least one solubility enhancing agent,
wherein the composition is prepared using a granulation technique.

2. The pharmaceutical composition according to claim 1, wherein the composition comprises about 30 mg to about 90 mg of ospemifene.

3. The pharmaceutical composition according to claim 1, wherein the ospemifene has an average particle size more than 25 microns.

4. The pharmaceutical composition according to claim 1, wherein the solubility enhancing agent is present intragranularly.

5. The pharmaceutical composition according to claim 1, wherein the composition is in the form of a tablet, a capsule, pellets, granules, a sachet, or sprinkles.

6. The pharmaceutical composition according to claim 1, wherein the pharmaceutically acceptable excipients comprise one or more of binders, fillers, complexing agents, enhancing agents, disintegrants, lubricants, glidants, sweetening agents, anti-tacking agents, or a combination thereof.

7. The pharmaceutical composition according to claim 1, wherein the solubility enhancing agent comprises a surface active agent comprising sodium lauryl sulfate, polysorbate 80, benzyl alcohol, sorbitan monolaurate, poloxamer 407 or combinations thereof.

8. The pharmaceutical composition according to claim 7, wherein the surface active agent comprises about 0.1 to about 6% by weight of the intra-granular composition.

9. The pharmaceutical composition according to claim 1 prepared by a process comprising:
a) preparing a solution or dispersion of ospemifene with one or more pharmaceutically acceptable excipients;
b) spraying the ospemifene solution or dispersion over a carrier bed to obtain granules;
c) drying the granules;
d) blending the dried granules with one or more pharmaceutically acceptable excipients;
e) lubricating the blend; and
f) processing the blend into the composition.

10. The pharmaceutical composition according to claim 1 prepared by a process comprising:
a) mixing ospemifene with one or more pharmaceutically acceptable excipients;
b) compacting the mixture to form slugs;
c) miffing the slugs to form a mixture of granules and fines;
d) granulating the milled mixture with one or more pharmaceutically acceptable excipients using a binder solution comprising at least one solubility enhancing agent;
e) drying the resulting granules;
f) sizing and milling the dried granules;
g) blending the granules with one or more pharmaceutically acceptable excipients;
h) compressing the blend into tablets; and
i) optionally coating the tablets.

11. A method of treatment or prevention of atrophy-related diseases or disorders in women, especially in women during or after the menopause, comprising orally administering to the subject the pharmaceutical composition according to claim 1.
